(19) United States
(12) Patent Application Publication
(10) Pub. No.: US 2026/0226001 A1
(43) Pub. Date: Aug. 6, 2026

(54) PROCESS FOR PREPARING AN N-GUANYLAMINO ACID

(71) Applicant: Evonik Operations GmbH, Essen (DE)

(72) Inventors: Juliane MERZ, Dortmund (DE); Jens SCHNEIDER, Bielefeld (DE); Kay MARIN, Borgholzhausen (DE); Frank JANKOWITSCH, Sassenberg (DE); Frank SCHNEIDER, Halle (DE); Alexander MAYER, Planegg (DE); Tobias DENZ, Frankfurt am Main (DE)

(73) Assignee: Evonik Operations GmbH, Essen (DE)

(21) Appl. No.: 19/153,224

(22) PCT Filed: Jan. 29, 2024
(86) PCT No.: PCT/EP2024/052034

§ 371 Date: Aug. 1, 2025

(30) Foreign Application Priority Data
Feb. 6, 2023 (EP) 23155154.0

(51) Int. Cl. C07C 277/08; A23K 20/142; A61K 31/198; C07C 279/14; C12P 13/04

(52) U.S. Cl. CPC C07C 277/08; A23K 20/142; A61K 31/198; C07C 279/14; C12P 13/04; C07B 2200/13

(57) ABSTRACT

The present invention relates to a process for preparing an N-guanylamino acid, e.g., GAA, comprising the steps of a) providing a liquid and/or solid mixture comprising an N-guanylamino acid, e.g., GAA, biomass and/or DNA, b) treating the mixture of step a) with an acidic solution and/or acidic agent, and c) isolating the N-guanylamino acid, e.g., GAA, from the mixture obtained in step b), characterized in that the step b) is performed at a pH lower than the pKa of the N-guanylaminoacetic acid of the mixture provided in step a), measured by means of a pH-electrode.

The present invention relates to a process for preparing an N-guanylamino acid, e.g., guanidinoacetic acid, N-guanylamino acid crystals with trapped guanine, and a method for supplementing an animal diet, wherein said diet is supplemented with the N-guanylamino acid, e.g., guanidinoacetic acid, obtained by the process according to the present invention and/or with the N-guanylamino acid, e.g., guanidinoacetic acid, crystals according to the present invention.

N-guanylamino acids are derivatives of amino acids with a guanidine group, which are obtainable by addition of cyanamide to the amino acid in question. The most important N-guanylamino acid is guanidinoacetic acid (GAA), also known as N-guanylglycine. It is an endogenous substance in animals and humans, which takes a central role in the biosynthesis of creatine. Creatine can be taken by the diet and/or be formed endogenously. Therefore, it is also used as a feed additive in animal nutrition (US 2011/257075 A1). Since it is a direct natural precursor of creatine, the supplementation of GAA allows for an optimal supply of creatine in the organism, which positively influences the energy transport in the muscle cells. Its biosynthesis proceeds from glycine and L-arginine. Guanidinoacetic acid is formed in the mammalian organism, primarily in the kidneys, by transferring the guanidine group of L-arginine by the enzyme L-Arg: Gly-amidinotransferase (AGAT) to the amino acid glycine. Starting from L-arginine, L-ornithine is thus produced, which is then metabolized in the urea cycle by carbamoylation to L-citrulline. In a further step, guanidinoacetic acid is methylated to creatine with S-adenosyl methionine by the enzyme guanidinoacetate N-methyltransferase (GAMT). Guanidinoacetic acid was first synthesized in 1861 by Adolph Strecker by addition of cyanamide to glycine in aqueous solution, specifically in weak ammoniacal aqueous solution (M. Strecker, compt. Rend. 1861, 52, 1212; cited in Ber. Chem. Ges. (now, Eur. J. Inorg. Chem.) 1908, 41, 4385). In later publications, guanidinoacetic acid was prepared from cyanamide and glycine in iso-propanol as solvent with sodium hydroxide as base (CN 102329250 A) or with sodium carbonate as base (CN101462983 A).

The published application CN113651726A discloses a process for preparing guanidinoacetic acid by reacting cyanamide with glycine in an alkali medium, followed by treating the thus obtained mixture with an acidic solution and/or acidic agent, and isolating the guanidinoacetic acid from that mixture.

The published application US 2022/0388948 A1 discloses a process for preparing guanidinoacetic acid, containing guanidinoacetic acid in a thermodynamically metastable crystal modification, by crystallizing it from an aqueous solution in the presence of at least one guanidine compound. This document also discloses the use of the GAA crystals thus obtained as feed supplement.

However, these methods have the significant drawback that the side-products melamine and dicyanamide are formed, which both have a negative effect on living organisms. Therefore, alternative methods for preparing N-guanylamino acids, e.g., GAA, were considered.

One of the alternative methods for preparing N-guanylamino acids, e.g., GAA, is by cultivating an N-guanylamino acid, e.g., GAA, producing microbial organism, for example, a genetically modified microorganism, or a microorganism, which is otherwise modified, e.g., by means of genome editing. This offers benefits over the chemical route, including the absence of the formation of the problematic side-products melamine and dicyanamide (EP 3839051 A, CN 111748506 A, and CN 113481139 A). For example, EP 3839051 A discloses a microorganism having an improved ability to produce L-arginine compared with the wildtype microorganism and/or having increased activities of an enzyme having the function of a carbamoylphosphate synthase compared to the respective enzymic activity in the wildtype microorganism and comprising at least one gene coding for a protein having the function of an L-arginine: glycine amidinotransferase. This document also discloses a method for the fermentative production of guanidinoacetic acid, comprising the steps of a) cultivating the said microorganism in a suitable medium under suitable conditions, and b) accumulating guanidinoacetic acid in the medium to form a guanidinoacetic acid containing fermentation broth.

However, the synthesis of N-guanylamino acids, e.g., GAA, via fermentation results in product particles comprising the biomass from the fermentation and/or the DNA (desoxyribose nucleic acid) of the genetically modified microorganism or of the otherwise modified microorganism, e.g., by genome editing, which was used in the fermentation, as impurity. Said biomass and/or DNA is trapped in the N-guanylamino acid, e.g., GAA crystals, which are formed in the fermentation process. Therefore, classical separation techniques such as filtration fail to remove or at least deplete this impurity.

Accordingly, there was still a need for a process for preparing N-guanylamino acids, such as guanidinoacetic acid, via fermentation without biomass and/or DNA as impurity or at least with reduced amounts of any of these impurities.

It was found that this problem is solved in that a liquid and/or solid mixture comprising N-guanylamino acid, e.g., GAA, biomass, and/or DNA, as obtained from a fermentation process, is treated with an acidic solution and/or acidic agent.

An object of the present invention is therefore a process for preparing an N-guanylamino acid, e.g., GAA, comprising the steps of
a) providing a liquid and/or solid mixture comprising an N-guanylamino acid, e.g., GAA, biomass and/or DNA,
b) treating the mixture of step a) with an acidic solution and/or acidic agent, and
c) isolating the N-guanylamino acid, e.g., GAA, from the mixture obtained in step b),
   characterized in that the step b) is performed at a pH lower than the pKa of the N-guanylaminoacetic acid of the mixture provided in step a), measured by means of a pH-electrode.

Preferably, the biomass and/or DNA in the mixture provided in step a) result(s) from the fermentative production of the N-guanylamino acid, e.g., GAA.

The process according to the present invention allows to remove or at least deplete the biomass and/or the DNA from any type of liquid and/or solid mixture comprising N-guanylamino acid, e.g., GAA, biomass and/or DNA. Said solid mixture can be either the aforementioned N-guanylamino acid crystals, e.g., GAA, in which the DNA of the microorganism used in the fermentation is trapped or it can be any other type of solid mixture comprising the fermentation product N-guanylamino acid, e.g., GAA, in non-crystalline form, biomass and/or DNA. Alternatively, in the case of a liquid mixture, the process according to the present invention allows to remove the DNA of the microorganism used in the fermentation process already from the N-guanylamino acid comprising fermentation broth.

In an embodiment of the process according to the present invention the liquid mixture of step a) comprises or consists of a fermentation broth, wherein said fermentation broth comprises the N-guanylamino acid; and the solid mixture of step a) comprises or consists of N-guanylamino acid crystals, wherein said crystals comprise biomass and/or DNA.

It was found that it is beneficial to perform the step b) at a pH lower than the pKa of the N-guanylamino acid of the mixture provided in step a). This leads to a protonation of the guanidine group of the N-guanylamino acid, which facilitates the dissolution of the N-guanylamino acid in question and the removal or at least depletion of any biomass and/or DNA from it. The pH at which step b) is performed, therefore depends on the individual N-guanylamino acid, e.g., GAA, provided in step a), and the medium in which step b) is performed. Preferably, step b) is performed in an aqueous medium. For example, when the N-guanylamino acid provided in step a) is guanidinoacetic acid, the step b) is performed at a pH lower than the pKa of guanidinoacetic acid, i.e., lower than approximately 2.9, measured by a pH-electrode. Depending on the individual composition of the mixture provided in step a) the pH at which step b) is performed may be different from the specific value of the pKa for a N-guanylamino acid, i.e., said pH may be different from 2.9 for guanidinoacetic acid. In the context of the present invention deviations from the pH values explicitly mentioned are still encompassed by the scope of the present invention, provided that they allow to achieve the benefits of the present invention.

In principle, the process according to the present invention is not subject to any limitations regarding a specific acidic agent in step b). Nevertheless, it was found that sulfuric acid is a suitable acidic for step b).

In a further embodiment of the process according to the present invention the acidic agent of step b) has a pKa ranging from −4 to +4, preferably ranging from −3 to +3.

In principle, the process according to the present invention and in particular step b) of said process is not subject to any limitations regarding a particular temperature. Nevertheless, it is preferred that the step b) is performed at elevated temperatures because an elevated temperature was found to be beneficial for the efficiency of step b), in particular with respect to the degree any biomass and/or DNA is removed or at least depleted from the guanylamino acid, e.g., GAA, comprising liquid or solid mixture and the time it takes for this.

In another embodiment of the process according to the present invention the step b) is performed at an elevated temperature.

In the context of the present invention an elevated temperature is in particular any temperature of 25° C. or more. The upper limit of the elevated temperature is given by a variety of factors, for example the boiling temperature of the solvent and the decomposition temperature of the organic substances other than the N-guanylamino acid, e.g., GAA, which are remainders from the fermentation process, e.g., sugars. For example, the step b) is performed at a temperature of from 25 to 90° C., 25 to 85° C., 25 to 80° C., 25 to 75° C., 25 to 70° C., 25 to 65° C., 25 to 60° C., 25 to 55° or 25 to 50° C.

In a preferred embodiment of the process according to the present invention the step b) is performed at a temperature of at least 25° C.

The synthesis of N-guanylamino acids via fermentation results in product particles in which the DNA (desoxyribose nucleic acid) of the genetically or otherwise modified microorganism used in the fermentation is trapped as impurity. The DNA is trapped in the GAA crystals, which are formed in the fermentation process. The treatment of the liquid and/or solid mixture comprising N-guanylamino acid, e.g., GAA, biomass and DNA with an acidic solution and/or acidic agent in step c) leads to a hydrolysis of said DNA, or at least a partial hydrolysis of said DNA.

In a further embodiment of the process according to the present invention the step b) comprises the acidic hydrolysis of DNA.

After the acidic treatment in step b) of the process according to the present invention the N-guanylamino acid, e.g., GAA, is isolated in step c) from the mixture obtained in step b). This isolation can be done by ultrafiltration, followed by concentration the thus obtained N-guanylamino acid, e.g., GAA, comprising permeate.

In yet another embodiment of the process according to present invention the step c) comprises the steps of
c1) removing the biomass from the mixture obtained in step b) by means of ultrafiltration to give a permeate, wherein said permeate comprises the N-guanylamino acid, e.g., GAA, and
c2) concentrating the permeate obtained in step c1).

The permeate obtained in step c1) may still comprise any non-desired remainders from the fermentation. Therefore, it is preferred that the permeate obtained in step c2) is not concentrated to dryness, e.g., the solvent is not completely removed under reduced pressure and/or elevated temperature. Rather, it is preferred that only a part of the medium of the permeate is removed in the concentration of step c2) and the thus concentrated N-guanylamino acid, e.g., GAA, comprising permeate from step c2) is fed to a crystallization. Preferably, said concentrating is done at elevated temperatures, i.e., temperatures of at least 25° C., preferably at least 30, 35 or 40° C., and/or under reduced pressure. While the reduced pressure is not subject to any limitations regarding a specific value, the upper limit of the elevated temperature is given by a variety of factors, for example the boiling temperature of the solvent and the decomposition temperature of the organic substances other than the N-guanylamino acid, e.g., GAA, which are remainders from the fermentation process, e.g., sugars. For example, the step c2) is performed at a temperature of from 40 to 100° C., 45 to 100° C., 50 to 100° C., 55 to 100° C., 60 to 100° C., 65 to 100° C., 70 to 100° C., 75 to 100° or 80 to 100° C.

In yet a further embodiment the process according to the present invention further comprises the step
d) feeding the isolated N-guanylamino acid, e.g., GAA, obtained in step c) to a crystallization.

In order to facilitate the crystallization in step c), it is preferred that the pH of the N-guanylamino acid, e.g., GAA, isolated in step c) or of the concentrated N-guanylamino acid, e.g., GAA, comprising permeate obtained in step c2) is set to a pH larger than the pKa of the N-guanylamino acid, e.g., GAA, and lower than the pKb of N-guanylamino acid, e.g., GAA, each measured by means of a pH-electrode. This leads to a deprotonation of the guanidine group of the N-guanylamino acid, e.g., GAA, which facilitates the crystallization of the N-guanylamino acid, e.g., GAA, in question. The pH at which step d) is performed, depends on the individual N-guanylamino acid, e.g., GAA, provided in step a), and the medium in which step b) is performed. Preferably, step d) is performed in an aqueous medium. For example, when the N-guanylamino acid provided in step a) is guanidinoacetic acid, the step d) is performed at a pH larger than the pKa of guanidinoacetic acid and lower than the pKb of guanidinoacetic acid, e.g., at a pH larger than approximately 2.9 and lower than approximately 10.91. Depending on the individual composition of the mixture provided in step a) the pH in step d) may be different from the specific value of the pKa and pKb for a N-guanylamino acid, e.g., GAA. In the context of the present invention deviations from the pH values explicitly mentioned are still encompassed by the scope of the present invention, provided that they allow to achieve the benefits of the present invention.

In a preferred embodiment of the process according to the present invention the step d) is performed at a pH larger than the pKa of the N-guanylamino acid, e.g., GAA, and lower than the pKb of the N-guanylamino acid, e.g., GAA, each measured by means of a pH-electrode.

After crystals of the N-guanylamino acid, e.g., GAA, are formed, said crystals can easily be separated from the mother liquor of the crystallization and be subject to drying.

In a further, preferred embodiment of the process according to the present invention the crystals obtained in step d) are separated by filtration and dried.

In principle, the process is not subject to any limitations regarding a specific N-guanylamino acid, e.g., GAA. However, the commercially most relevant N-guanylamino acid is guanidinoacetic acid.

In one embodiment of the present invention the N-guanylamino acid is guanidinoacetic acid.

Amino acids or derivatives thereof, such as N-guanylamino acids, e.g., guanidinoacetic acid, which were produced in fermentation processes, can be used in animal nutrition, e.g., in the supplementation of animal diets. The treatment of the liquid and/or solid mixture comprising N-guanylamino acid, biomass and/or DNA with an acidic solution and/or acidic agent in step c) leads to a hydrolysis of said DNA. As a result of this treatment the resulting N-guanylamino acid, e.g., GAA, is free or at least depleted from the DNA of the microorganism used in the fermentative production of the N-guanylamino acid, e.g., GAA. Guanine is a degradation product from the acidic hydrolysis that can be found in the N-guanylamino acid, e.g., guanidinoacetic acid. The resulting N-guanylamino acid, e.g., GAA, obtained by the process according to the present invention can be used without any limitations or requirements in the supplementation of animal diets. On the other hand, the presence of guanine in said N-guanylamino acid, e.g., GAA, crystals allows to identify if a N-guanylamino acid, e.g., GAA, was prepared or purified by the process according to the present invention.

Another object of the present invention is therefore N-guanylamino acid, e.g., GAA, crystals, wherein said crystals contain trapped guanine.

In an embodiment of the crystals according to the present invention the N-guanylamino acid is guanidinoacetic acid.

In a further embodiment of the crystals according to the present invention, the N-guanylamino acid, e.g., the guanidinoacetic acid, is obtained by the process according to the present invention.

A further object of the present invention is a method of supplementing an animal diet, wherein said diet is supplemented with the N-guanylamino acid, e.g., guanidinoacetic acid, obtained by the process according to the present invention and/or with the N-guanylamino acid, e.g., guanidinoacetic acid, crystals according to the present invention.

### EXAMPLE

After fermentation and inactivation of the fermentation broth, the pH of the broth was adjusted to 1.5 using 75% w/w of H₂SO₄ at a temperature of 50° C. The biomass was removed by means of ultrafiltration and the temperature of the broth was kept constant at 50° C. The resulting permeate was concentrated at 80° C. and used for the crystallization step. The permeate fed to the crystallization was adjusted to pH 8 using 25% w/w of NH₄OH. The formed crystals were separated by means of a suction filter and dried.

A part of the acidic and concentrated permeate was stored aside in a fridge over a period of ca. 2 weeks. The GAA content in said permeate was regularly determined by means of HPLC.

### Description of the HPLC Method:

Column: ThermoScientific HyperCarb 100×4.6 35007-104630

### Eluents:

### Eluent A:

2.3 g ammonium dihydrogenphosphate (NH₄H₂PO₄) and
2.6 g di-ammonium hydrogenphosphate ((NH₄)₂HPO₄)
dissolved in 2 L purified water

### Eluent B:

2.3 g ammonium dihydrogenphosphate (NH₄H₂PO₄) and
2.6 g di-ammonium hydrogenphosphate ((NH₄)₂HPO₄)
dissolved in 1 L purified water and mixed with 1 L acetonitrile

The following gradient was used in the HPLC method:

| Time [min] | Flow [mL/min] | % Eluent A | % Eluent B |
|---|---|---|---|
| 0.000 | Equilibration | | |
| 0.000 | Start Run | | |
| 0.000 | 1.000 | 100.0 | 0.0 |
| 8.000 | 1.000 | 90.0 | 10.0 |
| 10.000 | 1.000 | 60.0 | 40.0 |
| 11.000 | 1.000 | 60.0 | 40.0 |
| 11.100 | 1.000 | 100.0 | 0.0 |
| 13.000 | 1.000 | 100.0 | 0.0 |

| | |
|---|---|
| 14.000 | Stop Run |

The results of the determination of the GAA content are summarized in the table below:

| Date | GAA content [g/kg] |
|---|---|
| 1 Apr. 2022 | 120.9 |
| 7 Apr. 2022 | 118.5 |
| 12 Apr. 2022 | 119.9 |
| 13 Apr. 2022 | 119.3 |

The results show that, within the scope of measurement accuracy, the GAA content was constant over the 2-week period. This means that there was no degradation of the GAA in the acidic and concentrated permeate.

1. A process for preparing an N-guanylamino acid, the process comprising:
a) providing a liquid and/or solid mixture comprising an N-guanylamino acid, biomass and/or DNA,
b) treating the mixture of a) with an acidic solution and/or acidic agent, and
c) isolating the N-guanylamino acid from the mixture obtained in b),
wherein the b) is performed at a pH lower than a pKa of the N-guanylaminoacetic acid of the mixture provided in a), measured by a pH-electrode.

2. The process according to claim 1, wherein the liquid mixture in a) comprises a fermentation broth comprising the N-guanylamino acid, and wherein the solid mixture in a) comprises N-guanylamino acid crystals comprising biomass and/or DNA.

3. The process according to claim 1, wherein the acidic agent in b) has a pKa of from −4 to +4.

4. The process according to claim 1, wherein b) is performed at an elevated temperature.

5. The process according to claim 1, wherein b) is performed at a temperature of at least 25° C.

6. The process according to claim 1, wherein b) comprises acidic hydrolysis of DNA.

7. The process according to claim 1, wherein c) comprises:
c1) removing the biomass from the mixture obtained in b) by ultrafiltration to obtain a permeate, wherein the permeate comprises the N-guanylamino acid, and
c2) concentrating the permeate obtained in c1).

8. The process according to claim 1, further comprising:
d) feeding the isolated N-guanylamino acid obtained in c) to a crystallization.

9. The process according to claim 8, wherein d) is performed at a pH larger than the pKa of the N-guanylamino acid and lower than a pKb of the N-guanylamino acid, each measured by a pH-electrode.

10. The process according to claim 8, wherein crystals obtained in d) are separated from a mother liquor of the crystallization by filtration and dried.

11. The process according to claim 1, wherein the N-guanylamino acid is guanidinoacetic acid.

12. N-guanylamino acid crystals, wherein the crystals contain comprise trapped guanine.

13. The N-guanylamino acid crystals according to claim 12, wherein the N-guanylamino acid is guanidinoacetic acid.

14. The N-guanylamino acid crystals according to claim 12, wherein the N-guanylamino acid is obtained by a process comprising:
a) providing a liquid and/or solid mixture comprising an N-guanylamino acid, biomass and/or DNA,
b) treating the mixture of a) with an acidic solution and/or acidic agent, and
c) isolating the N-guanylamino acid from the mixture obtained in b),
wherein the b) is performed at a pH lower than a pKa of the N-guanylaminoacetic acid of the mixture provided in a), measured by a pH-electrode.

15. A method of supplementing an animal diet, comprising supplementing the animal diet with the N-guanylamino acid obtained by the process of claim 1.

16. A method of supplementing an animal diet, comprising supplementing the animal diet with the N-guanylamino acid crystals of claim 12.
